(19) United States
(12) Patent Application Publication
(10) Pub. No.: US 2026/0360538 A1
(43) Pub. Date: Nov. 12, 2026

(54) AI-Powered Diagnosis Verification and Blockchain-Audited Reevaluation System

(71) Applicant: Mohamed Elsabrouty, Dallas, TX (US)

(72) Inventor: Mohamed Elsabrouty, Dallas, TX (US)

(21) Appl. No.: 19/204,533

(22) Filed: May 10, 2025

(51) Int. Cl. G16H 50/20; G16H 10/60; G16H 50/30

(52) U.S. Cl. CPC G16H 50/20; G16H 50/30; G16H 10/60

(57) ABSTRACT

A system for verifying medical diagnoses using artificial intelligence. Upon detecting a conflict between AI-generated diagnostics and clinician input, a logic-gate module triggers a lock-out state that suspends data transmission until reconciliation is provided via patient reevaluation or recorded justification. Diagnostic actions are logged as cryptographic hashes on a permissioned blockchain. An insurance gateway validates these hashes as a condition for claim approval. This architecture ensures diagnostic accountability and automated audit transparency.

### BACKGROUND

Diagnostic errors remain a significant cause of medical harm. Current Electronic Health Records (EHRs) lack real-time AI support, physician override tracking, and transparent audit access for insurance providers. No current system effectively integrates AI diagnosis validation, blockchain logging, and automated insurer access.

### SUMMARY

The present invention provides a technical solution for diagnostic accountability by integrating an AI-driven verification engine with a decentralized blockchain architecture. The system comprises a logic-gate module that monitors for discrepancies between clinician-input diagnoses and AI-generated diagnostic inferences. Upon detection of a discrepancy, the logic-gate module triggers a mandatory lock-out state within the clinical workflow, electronically suspending the transmission of medical claims and prescriptions to external servers. This technical safeguard ensures that diagnostic conflicts are reconciled—either through reevaluation or recorded justification—prior to the execution of downstream data transactions. By anchoring physician justifications and reconciliation actions as cryptographically signed hashes in a permissioned blockchain ledger, the system provides a tamper-proof audit trail that enhances data integrity and prevents the propagation of erroneous clinical data.

### BRIEF DESCRIPTION OF THE DRAWINGS

FIG. 1 shows the overall system flow from a doctor's diagnosis to AI evaluation, physician action, and blockchain logging.

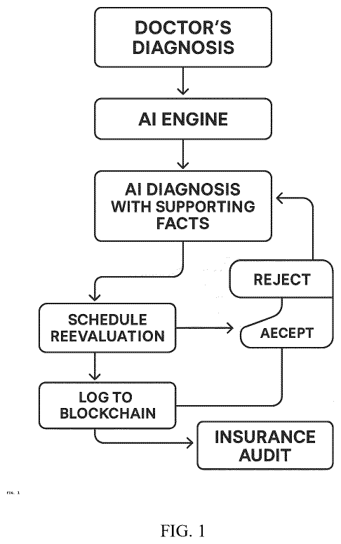

FIG. 2 illustrates the physician interface displaying AI diagnosis, supporting facts, and the options to accept or reject.

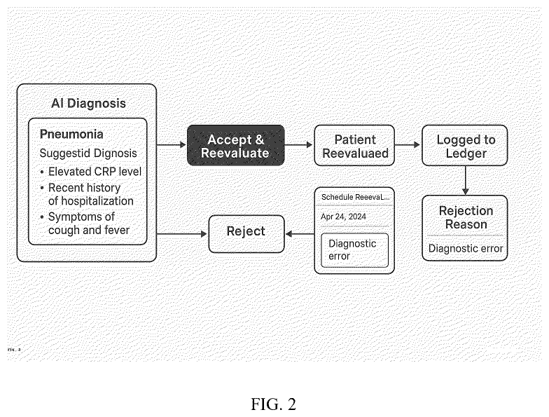

FIG. 2A shows a rescheduling interface displayed to the physician after accepting an AI diagnosis for reevaluation.

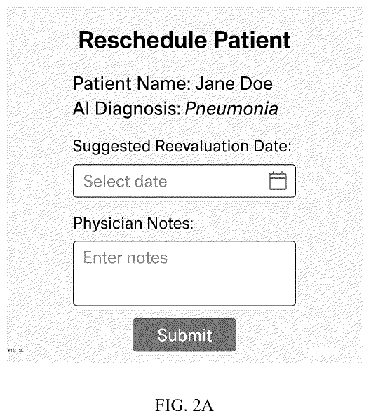

FIG. 3 presents a ledger-style audit trail with timestamps, decisions, and blockchain hashes for insurer access.

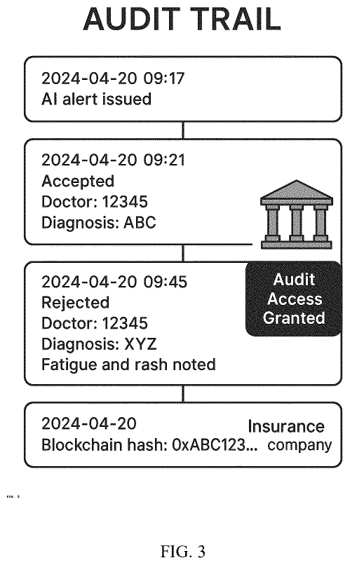

FIG. 4 outlines a clinical logic flowchart guiding AI decision-making based on symptoms, labs, and prior conditions.

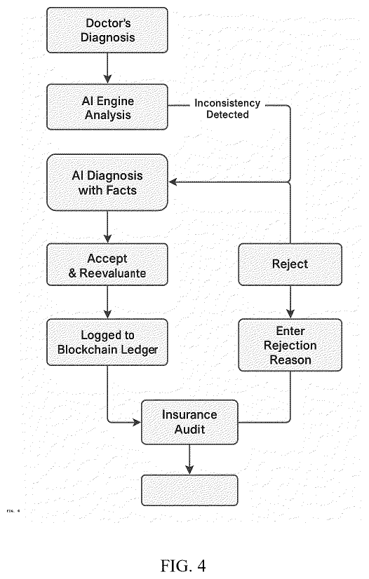

FIG. 5 displays the data pipeline from EHR extraction to AI analysis, logging, and secure insurer access.

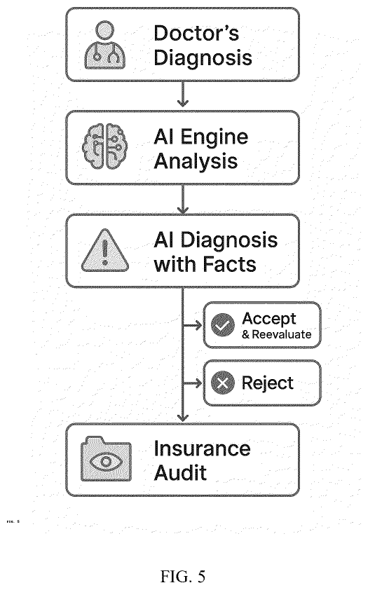

### DETAILED DESCRIPTION

The system comprises an AI Engine, a Physician Decision Interface, a Blockchain Logger, and an Insurance Access Gateway. When a doctor enters a diagnosis, the AI flags discrepancies using clinical data.

The system integrates a logic-gate module that enters a lock-out state upon detecting a discrepancy. This state prevents the electronic transmission of medical claims or prescriptions until the physician provides a reconciliation reason or schedules a reevaluation. This ensures that no unvetted or conflicting diagnosis moves to the billing phase without an explicit human-in-the-loop justification.

Physician actions are recorded on a permissioned blockchain, where each response is hashed and signed. The Insurance Access Gateway validates the presence of a unique blockchain-anchored justification hash as a mandatory condition for claim reimbursement.

### TECHNICAL IMPLEMENTATION

The AI Engine comprises a fine-tuned large language model (LLM) and a rules-based decision layer trained on clinical datasets. Inputs include structured (labs, vitals) and unstructured (clinical notes) data, processed using HL7 FHIR APIS.

The Disagreement Alert Module calculates confidence scores and triggers alerts when the physician diagnosis deviates from the AI recommendation.

The Physician Decision Interface is built as a secure web application allowing decision entry, scheduling, or rejection logging.

Blockchain logging is implemented using Hyperledger Fabric. Each physician response (acceptance, rescheduling, or rejection reason) is hashed, signed, and stored immutably in a permissioned ledger.

The Insurance Access Gateway allows audit-triggered read access for claim validation via API with OAuth-based permissions.

The system runs on a containerized cloud infrastructure using Docker and Kubernetes, with audit trails backed up in redundant, encrypted nodes.

### GLOSSARY

AI Engine: Artificial Intelligence system combining LLMs and clinical rule engines for medical inference.

FHIR: Fast Healthcare Interoperability Resources-a standard for healthcare data exchange.

Hyperledger Fabric: A permissioned blockchain framework used for secure transaction logging.

Blockchain Ledger: A distributed ledger for storing immutable records of physician actions.

Disagreement Alert: Notification triggered when AI and physician diagnoses differ significantly.

Rejection Reason Capture: Interface element requiring physician justification when rejecting AI suggestions.

### PRIOR ART COMPARISON

IBM Watson for Health provides diagnostic suggestions but lacks physician override tracking and blockchain accountability.

MediLedger offers blockchain for pharma supply chain, not real-time clinical decision logging.

No known system integrates explainable AI with override justification logging, real-time alerts, and permissioned insurer audit access. This invention uniquely addresses diagnostic accountability by fusing AI, clinical workflows, and blockchain compliance.

### PUBLIC SUMMARY

This system helps doctors make safer decisions by showing AI-generated diagnoses and alerts if the diagnosis seems incorrect. If the doctor agrees, they can reevaluate the patient and schedule a follow-up. If they disagree, they must enter a reason. All decisions are recorded securely on a blockchain. Insurance companies can access these records if needed to verify medical decisions before paying claims. This helps reduce errors, improve transparency, and ensure fairness in billing and care.

1. A computer-implemented diagnosis-verification system comprising: one or more processors; a memory storing instructions executable by the one or more processors; an artificial-intelligence engine configured to analyze patient data and generate a suggested diagnosis; a disagreement-alert module configured to detect a discrepancy between the suggested diagnosis and a physician-entered diagnosis; a logic-gate module configured, in response to detecting the discrepancy, to enter a lock-out state that prevents electronic transmission of a medical claim or prescription to an external server; a secure physician decision interface configured to receive either a physician justification or reevaluation scheduling information in response to the discrepancy; and a blockchain logger configured to hash, digitally sign, and store the received physician justification or reevaluation scheduling information in a permissioned blockchain ledger, wherein the logic-gate module is configured to clear the lock-out state only after receipt of the physician justification or the reevaluation scheduling information.

2. The system of claim 1, wherein the disagreement-alert module is configured to generate an alert upon detecting the discrepancy.

3. The system of claim 1, wherein the blockchain logger is configured to immutably store physician responses, physician justifications, and reconciliation actions in the permissioned blockchain ledger.

4. The system of claim 1, wherein the secure physician decision interface is configured, upon receipt of an acceptance selection, to present a rescheduling interface and receive a future appointment date for a reevaluation session.

5. The system of claim 1, wherein the secure physician decision interface is configured, upon receipt of a rejection selection, to require the physician justification before the logic-gate module clears the lock-out state.

6. The system of claim 1, wherein the blockchain logger is configured to record an acceptance selection or a rejection selection using a unique cryptographic hash in the permissioned blockchain ledger.

7. The system of claim 1, wherein the secure physician decision interface is configured to display facts extracted by the artificial-intelligence engine and controls for receiving an acceptance selection or an override.

8. The system of claim 1, wherein the artificial-intelligence engine comprises a large language model and a rules-based clinical reasoning engine.

9. The system of claim 1, wherein the disagreement-alert module is configured to calculate a confidence score and apply a configurable clinical-risk threshold to determine whether to generate the alert.

10. A computer-implemented method for verifying a physician diagnosis, the method comprising: receiving patient data and a physician-entered diagnosis; generating, by an artificial-intelligence engine, a suggested diagnosis based on the patient data; detecting a discrepancy between the physician-entered diagnosis and the suggested diagnosis; in response to detecting the discrepancy, placing a logic-gate module in a lock-out state that prevents electronic transmission of a medical claim or prescription to an external server; receiving, through a secure physician decision interface, either a physician justification or reevaluation scheduling information; hashing and digitally signing the received physician justification or reevaluation scheduling information; storing the hashed and digitally signed physician justification or reevaluation scheduling information in a permissioned blockchain ledger; and clearing the lock-out state only after receiving the physician justification or the reevaluation scheduling information.

11. The method of claim 10, wherein receiving the reevaluation scheduling information comprises receiving a future appointment date, and wherein receiving the physician justification comprises requiring a rejection reason before clearing the lock-out state.

12. The method of claim 10, further comprising storing a physician decision, the physician justification, and a digital signature in the permissioned blockchain ledger.

13. A healthcare information system comprising: one or more processors; a memory storing instructions executable by the one or more processors; an artificial-intelligence module configured to generate a suggested diagnosis from electronic health record data; a conflict-detection engine configured to detect a discrepancy between the suggested diagnosis and a physician-entered diagnosis; a secure physician user interface configured to receive a physician response to the discrepancy; a blockchain logger configured to generate a blockchain-anchored justification hash from the physician response and store the physician response in a permissioned blockchain ledger; and an insurance payout gateway configured to validate the presence of the blockchain-anchored justification hash before electronically approving an insurance claim.

14. The healthcare information system of claim 13, wherein the insurance payout gateway is configured to use validation of the blockchain-anchored justification hash as a mandatory condition for electronically approving the insurance claim.

15. The healthcare information system of claim 13, further comprising a secure Health Level Seven Fast Healthcare Interoperability Resources application programming interface configured to extract the electronic health record data and an OAuth-protected application programming interface configured to provide claim-validation access based on the physician response.
